(19) United States
(12) Patent Application Publication
(10) Pub. No.: US 2016/0271399 A1
(43) Pub. Date: Sep. 22, 2016

(54) Feedthrough of an Implantable Electronic Medical Device and Implantable Electronic Medical Device

(71) Applicant: BIOTRONIK SE & Co. KG, Berlin (DE)

(72) Inventor: Marcel Starke, Eichwalde (DE)

(21) Appl. No.: 15/054,496

(22) Filed: Feb. 26, 2016

Related U.S. Application Data
(60) Provisional application No. 62/135,709, filed on Mar. 20, 2015.

(51) Int. Cl. A61N 1/375

(52) U.S. Cl. CPC A61N 1/3754

(57) ABSTRACT

A feedthrough of an implantable electronic medical device, including a housing, an insulating body, a feedthrough flange surrounding the insulating body, and at least one connecting element penetrating the insulating body for externally connecting an electric or electronic component of the device, in particular multiple connecting elements, wherein a low-temperature hard solder connection or soft solder connection is provided between the insulating body and the feedthrough flange and/or between the insulating body and the, or at least one, connecting element and/or between the insulating body and the housing, the connection being formed in particular at a temperature of 900° C. or less, preferably less than 450° C., and still more preferably less than 400° C.

### CROSS-REFERENCE TO RELATED APPLICATIONS

This patent application claims the benefit of co-pending U.S. Provisional Patent Application No. 62/135,709, filed on Mar. 20, 2015, which is hereby incorporated by reference in its entirety.

### TECHNICAL FIELD

The present invention relates to a feedthrough of an implantable electronic medical device, comprising an insulating body, a feedthrough flange surrounding the insulating body, and at least one connecting element penetrating the insulating body for externally connecting an electric or electronic component of the device, in particular multiple connecting elements. It further relates to an implantable electronic medical device containing such a feedthrough.

### BACKGROUND

Such implantable devices have been widely used for quite some time, in particular, as cardiac pacemaker or implantable cardioverters (specifically defibrillators), to name a few. However, they can also involve a less complex apparatus, such as, for example, an electrode lead or sensor line or also a cochlea implant.

The majority of implantable electromedical devices that are significant in practice are intended to deliver electric pulses to excitable body tissue via suitably positioned electrodes. So as to carry out this function, electronic/electric functional units are accommodated in the housing of the device for generating the pulses and for suitably controlling the pulse generation, and electrodes or connections for at least one electrode lead are provided directly on the outside of the device, in the distal end section of which the electrodes for transmitting the pulse to the tissue are accommodated. The electronic/electric functional units in the housing interior are to be connected to the outer electrodes or electrode lead connections in a way that, under special conditions of the implanted state, ensures absolutely and permanently reliably function.

In particular, feedthroughs are known, the basic and insulating body of which is essentially made of ceramic material or glass, wherein multi-layer or multi-piece attachments using metals or metal oxides have also been developed and are used. Such known feedthroughs largely meet the demands placed on them. However, the thermal coefficients of expansion must be taken into consideration in the material selection of the insulation ceramic/glass, metal solder or glass solder, metal pin and metal flange, so as to be able to ensure sufficient tightness over the intended service life.

In the conventional design (metal flange—solder—insulating ceramic—solder—metal pin), the effect of thermal coefficients of expansion that are not matched can primarily be felt during cooling from the solder temperature and when welding the feedthrough into the housing. This can result in mechanical tensile stresses, which can lead to material separation and, consequently, to possible leakage of the feedthrough. The ceramic and metallic components used in conventional feedthroughs are connected to each other by the solder material; during uneven expansion/shrinkage of the components among each other, including the solder, due to heating/cooling processes, the ensuing relative longitudinal changes cause corresponding mechanical stresses.

A hermetically sealed feedthrough structure is known from European Patent No. EP 2 232 646, which includes a multi-piece basic or insulating body in combination with sealing (not structural) polymer layers. Such a feedthrough is extremely complex to produce in terms of the required work and test steps, and also in terms of the prefabrication, storage and feeding of many different parts.

U.S. Pat. No. 7,064,270 also describes a feedthrough having a multi-piece design, which was developed specifically for an electrode lead and can comprise multiple components made of plastic material or provided with a plastic coating.

An electronic device is known from European Publication No. EP 2 388 044 which has a feedthrough having a basically simple design made of a liquid crystal polymer. No details of the device design are disclosed in this published prior art.

It is also known to carry out brazing processes for bonding titanium and nickel parts, for example, using eutectic solders, and thereby reduce the thermal stresses, and the problems resulting therefrom, accompanying weld joints or high-temperature soldering process; see N. Weyrich et. al. “Joining of Titanium and Nickel at Temperatures Below 450° C.”, Brazing, High Temperature Brazing and Diffusion Bonding, Löt 2013, pg. 22. It is further known to use Au alloy solders having a low melting point for soldering capacitive filters into feedthroughs of implantable medical devices; see U.S. Pat. No. 5,870,272 or U.S. Pat. No. 6,031,710 in this regard.

The present invention is directed toward overcoming one or more of the above-mentioned problems.

### SUMMARY

It is an object of the present invention to provide an improved implantable electromedical device, which is cost-effective to produce and highly reliable.

At least this object is achieved by a feedthrough having the features of claim 1. Advantageous refinements of the inventive concept are the subject matter of the dependent claims. Moreover, a corresponding implantable electronic medical device is also disclosed.

The present invention is based on the deliberation to implement the heating and cooling steps, which are always critical for a permanently reliable function of the feedthrough, with considerably smaller temperature differences and, thereby, at least significantly reduce the aforementioned problems. Added to this is the deliberation that reduced maximal process temperatures offer the potential for the use of materials that are less temperature-resistant and generally provide greater degrees of freedom in the design of the feedthrough. This leads to the deliberation to provide a low-temperature hard solder connection or soft solder connection between the insulating body and the feedthrough flange and/or between the insulating body and the, or at least one, connecting element and/or between the insulating body and the housing, the connection being formed in particular at a temperature of 900° C. or less, preferably less than 450° C., and still more preferably less than 400° C.

In one embodiment of the present invention, the feedthrough comprises the low-temperature hard solder connection formed of a eutectic gold alloy solder melting below 900° C., in particular below 450° C., and further particularly below 400° C., such as, for example, Au80Sn20, Au81Si19, Au94Sn6, or the like.

It is provided in further embodiments of the present invention that the feedthrough comprises a constituent or a region that has limited temperature stability up to a temperature of only 1,050° C., in particular only up to 950° C., or less.

It is provided in one design of these embodiments that the constituent or region having limited temperature stability comprises a metal ceramic composite.

In one variant of this embodiment, the constituent having limited temperature stability comprises an electronic system produced in LTCC technology, in particular, a system of connecting elements.

In further embodiments, the insulating body comprises at least one plastic component. In one embodiment, it is provided that the plastic component is a plastic injection-molded part or a plastic coating of a ceramic or glass part. Moreover, the plastic component can comprise a filling having non-organic and non-metallic particles, in particular ceramic and/or glass particles.

Further embodiments, features, aspects, objects, advantages, and possible applications of the present invention could be learned from the following description, in combination with the Figures, and the appended claims.

### DESCRIPTION OF THE DRAWINGS

Advantages and functional characteristics of the invention will additionally become apparent from the description of exemplary embodiments based on the Figures. In the drawings:

FIG. 1 shows a schematic, partially cut illustration of an implantable electromedical device of the present invention.

FIG. 2 shows a schematic cross-sectional illustration (partial view) of one exemplary embodiment of the present invention.

FIG. 3 shows a schematic cross-sectional illustration (partial view) of a further exemplary embodiment of the present invention.

FIG. 4 shows a schematic cross-sectional illustration (partial view) of a further exemplary embodiment of the present invention

### DETAILED DESCRIPTION

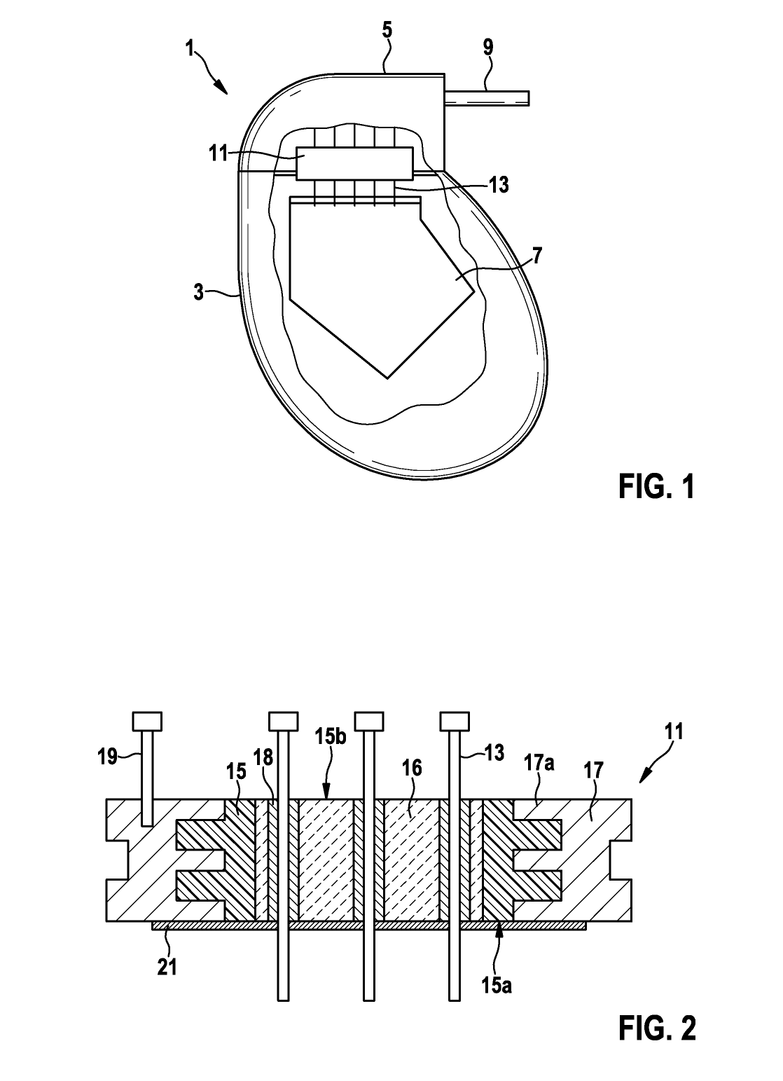

FIG. 1 shows a cardiac pacemaker 1 comprising a pacemaker housing 3 and a header 5, in the interior of which a printed circuit board (PCB) 7 is disposed, in addition to other electronic components, and to the line connection of which disposed in the header (not shown) an electrode lead 9 is connected. A feedthrough 11 provided between the device housing 3 and the header 5 comprises a plurality of connecting pins 13. At one end, the connecting pins 13 are placed through an appropriate borehole in the printed circuit board 7 and are soft-soldered thereto.

Using the same reference numerals for functionally equivalent parts as in FIG. 1, FIG. 2 shows a composition of a feedthrough 11 by way of example. This comprises an annular plastic base body 15 generated as an injection-molded part and an inner, disk-shaped ceramic base body 16, inserted into a feedthrough flange 17 formed by way of powdered metal injection molding (MIM technology). On the inner circumference, the flange 17 carries multiple annular extensions 17a projecting inward into the material of the plastic main body 15 molded directly into the flange 17. These ensure multiple interlocking with the plastic material and, thus, a hermetically sealed connection between the outer plastic base body 15 and the flange 17. The outer circumference of the inner ceramic base body 16 has no such extensions in the illustration in the Figure; however, in practice, such extensions can also be provided there and create a similar effect as on the flange 17.

Multiple connecting pins 13 penetrate the inner ceramic base body 16. They are inserted into the same in each case by bonding by way of a soft solder connection 18 made of, for example, a eutectic gold alloy solder. Due to the use of a low-temperature solder connection, it becomes possible to solder the connecting pins 13 into the inner ceramic base body 16 regardless of whether the surrounding outer plastic base body 15 has limited temperature resistance and would not withstand the temperatures required with conventional brazing methods.

A ground pin 19 is additionally inserted into the flange 17 (again, soldered in or generated simultaneously in an MIM process). A barrier layer 21 covering the two base bodies 15, 16 on the feedthrough surface 15a improves the diffusion resistance of the feedthrough with respect to gaseous or liquid constituents of the surroundings in which the device is used.

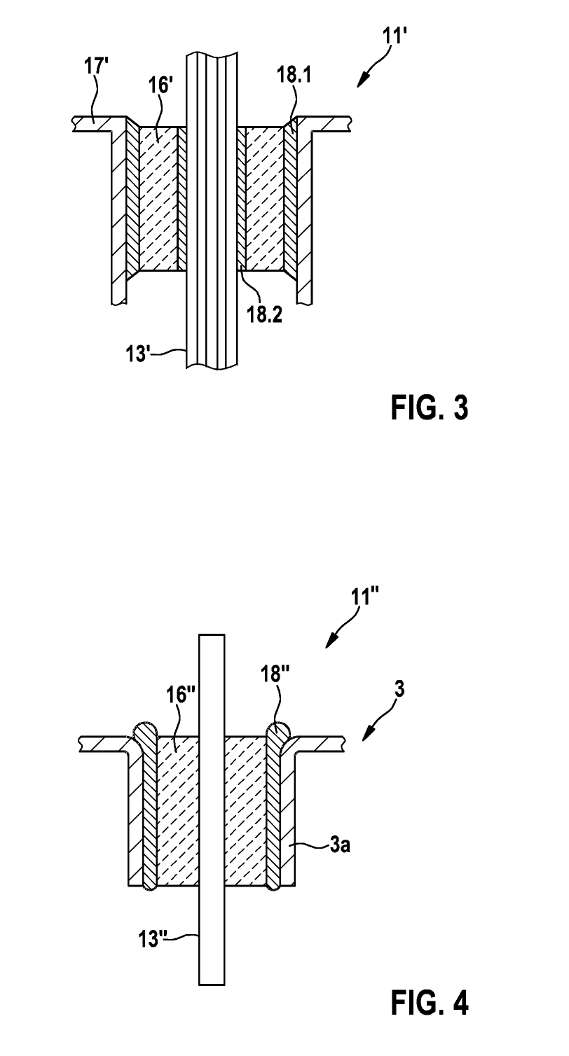

FIG. 3 shows a highly simplified schematic illustration of a further embodiment of a feedthrough 11′, in which a ceramic insulating body 16′ is connected on the outer wall thereof via a low-temperature hard solder connection 18.1 to a cold-formed feedthrough flange 17′, and in which a low-temperature hard solder connection 18.2 is provided in the interior of the base body for embedding, in a hermetically sealed manner, a relatively heat-sensitive connecting element system 13′ produced in LTCC technology. A low-temperature hard solder connection in the present invention shall be understood to mean such a connection which can be generated under a free atmosphere, which is to say not under vacuum or under protective gas. Here as well, the use of a eutectic low-temperature solder not only allows stress loads to be reduced and the general reliability to be increased, but above all also enables a novel design principle of the feedthrough.

FIG. 4, again in a highly schematic illustration, shows a further exemplary feedthrough 11″, in which a ceramic insulating body 16″, which here surrounds a single connecting pin 13″, is soldered directly to a bent section 3a of an implant housing 3 by way of a low-temperature hard solder connection 18″.

The present invention can also be carried out in a plurality of modifications of the examples shown here and of aspects of the present invention that are pointed out above.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A feedthrough of an implantable electronic medical device, comprising:
a housing;
an insulating body;
a feedthrough flange surrounding the insulating body; and
at least one connecting element penetrating the insulating body for externally connecting an electric or electronic component of the device, in particular a plurality of connecting elements,
wherein a low-temperature hard solder connection or soft solder connection is provided between the insulating body and the feedthrough flange and/or between the insulating body and the, or at least one, connecting element and/or between the insulating body and the housing, the connection being formed at a temperature of 900° C. or less.

2. The feedthrough according to claim 1, wherein the low-temperature hard solder connection is formed of a eutectic gold alloy solder melting below 900° C. and comprising Au80Sn20, Au81Si19, or Au94Sn6.

3. The feedthrough according to claim 1, further comprising a constituent or a region that has limited temperature stability up to a temperature of only 1,050° C. or less.

4. The feedthrough according to claim 3, wherein the constituent or region having limited temperature stability comprises a metal ceramic composite.

5. The feedthrough according to claim 3, wherein the constituent having limited temperature stability comprises an electronic system produced in LTCC technology and comprising a system of connecting elements.

6. The feedthrough according to claim 3, wherein the insulating body comprises at least one plastic component.

7. The feedthrough according to claim 6, wherein the plastic component is a plastic injection-molded part or a plastic coating of a ceramic or glass part.

8. The feedthrough according to claim 6, wherein the plastic component comprises a filling having non-organic and non-metallic particles comprising ceramic and/or glass particles.

9. An implantable electronic medical device comprising a feedthrough according to claim 1, wherein the implantable electronic medical device is designed as a cardiac pacemaker, implantable cardioverter or cochlear implant.

10. The feedthrough according to claim 1, wherein the connection is formed at a temperature of 450° C. or less.

11. The feedthrough according to claim 10, wherein the low-temperature hard solder connection is formed of a eutectic gold alloy solder melting below 450° C. and comprising Au80Sn20, Au81Si19, or Au94Sn6.

12. The feedthrough according to claim 1, wherein the connection is formed at a temperature of 400° C. or less.

13. The feedthrough according to claim 12, wherein the low-temperature hard solder connection is formed of a eutectic gold alloy solder melting below 400° C. and comprising Au80Sn20, Au81Si19, or Au94Sn6.

14. The feedthrough according to claim 1, further comprising a constituent or a region that has limited temperature stability up to a temperature of only 950° C. or less.
